(19) United States
(12) Patent Application Publication
(10) Pub. No.: US 2006/0074077 A1
(43) Pub. Date: Apr. 6, 2006

(54) Pharmaceutical composition for the treatment of athetoid movement

(76) Inventor: Yang Soo Lee, Susung-gu (KR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/244,906

(22) Filed: Oct. 5, 2005

(30) Foreign Application Priority Data
Oct. 5, 2004 (KR) 2004-78958

(51) Int. Cl. A61K 31/5513; A61K 31/551

(52) U.S. Cl. 514/221

(57) ABSTRACT

A pharmaceutical composition for the treatment of athetoid movement comprising clonazepam or pharmaceutically acceptable salts thereof as an active ingredient are provided, and are useful for the treatment of athetoisis without any tolerance and adverse effect.

### FIELD OF THE INVENTION AND THE RELATED PRIOR ART

The present invention relates to a pharmaceutical composition for the treatment of athetoid movement, comprising clonazepam or pharmaceutically acceptable salts thereof as an active ingredient.

Athetoid movement, which is often observed in individuals suffering from athetoid cerebral palsy, was named in the sense of involuntary movement of the body against patient's will not to move. Such athetoid movement is manifested violently in the face, neck and arms and revealed slightly in the legs.

Athetoid cerebral palsy is developed due to lesion of basal ganglia, and is characterized by involuntary movement as described above, which affects balance of the body, impairs movements of the hands and feet, shows symptom of turning continuously the neck and thus gives rise to degenerative alteration of cervical spine in its early stage and further leads to injury to the spinal cord and eventually develops even to patients who can walk various complications including gait disorders. Said athetoid movement, when being revealed in the facial or tongue muscle, shows symptoms such as speech disturbances, facial distortions and flowing sputum. Therefore, for patients with athetoid movement, reduction of athetoid movement is very important for improving coordination of the hands and feet and preventing from developing complications.

On the other hand, clonazepam (C₁₅H₁₀ClN₃O₃), which is white or pale yellow, odorless crystal or crystalline powder with a melting point of about 240° C., has been used as medicament of epilepsy. However, it has not been reported that clonazepam can be effectively used for the treatment of athetoid movement.

### TECHNICAL TASK TO BE ACHIEVED BY THE INVENTION

Therefore, purpose of the present invention is to provide new use of clonazepam for the treatment for athetoid movement.

### CONSTITUTION OF THE INVENTION

The present invention provides a pharmaceutical composition for the treatment of athetoid movement, comprising clonazepam or pharmaceutically acceptable salts thereof as an active ingredient.

The present invention relates to a new indication of clonazepam, the treatment of athetoid movement.

Clonazepam (5-(o-chlorophenoxy)-1,3-dihydro-7-nitro-2H-1,4-benzodiazepine-2-one) has been used as medicament of epilepsy, especially, epileptic seizure and partial seizure (focal seizure), primary and secondary generalized grand mal seizure and infantile epilepsy (especially, typical * atypical absence seizure), and is on sale in the name of Klonopin™ or Rivotril®.

Athetoid movement means that slow, staggering, serpentine and involuntary movement, particularly arising in the limbs. This dyskinesia is manifested also in the axial muscle and the cephalic -cervical muscle, and causes continuous mobile spasm due to mixed movement together. These symptoms of athetoid movement are manifested mainly in cerebral palsy due to kernicterus, infantile meningitis, and are also manifested by lesion of basal ganglia or trauma.

Said clonazepam may be used alone or provided in the form of pharmaceutically acceptable salts thereof for the treatment for athetoid movement. Said pharmaceutically acceptable salts include inorganic alkalis such as alkali metal hydroxides, alkali metal bicarbonates and alkali metal carbonates and organic alkalis such as primary, secondary and tertiary amine, amino acids.

The method of treatment for athetoid movement using a pharmaceutical composition in accordance with the present invention is as follows.

First, for rapid therapy of athetoid movement, the usual dose of the active ingredient of a pharmaceutical composition of the present invention is 0.005 to 0.03 mg/kg body weight/day, divided three times a day. If drowsiness or muscle wasting is shown, the frequency of administration is decreased from three times a day to one or two times a day.

For slow therapy of athetoid movement, the usual dose of the active ingredient of a pharmaceutical composition of the present invention is 0.005 to 0.01 mg/kg body weight/day, one time a day, and then the dose is increased by 20 to 40% every three days or every seven days.

In this case, the dose of the active ingredient shall not exceed 0.03 mg/kg body weight/day, which is less than the dose for the treatment of epilepsy, and therefore athetoid movement can be treated effectively without any adverse effect such as tolerance, drowsiness or muscle wasting.

For reference, when clonazepam is used for the treatment of epilepsy in adult, the starting does is daily divided in the same three doses, not exceeding 1.5 mg/day, and then the dose is increased by 0.5 mg every three days, and the maintenance dose is 3 to 6 mg/day.

That is, usual drugs based on benzodiazepines have the problem that they relax skeletal muscles slightly but exert too strong sedation at the effective dose, which relieves uneasiness, anxiety or emotional stress etc., and thus they have to be used only restrictively. However, clonazepam of the present invention relaxes muscles at a low dose, which does not exert sedation. By administering clonazepam to patients with athetoid movement taking into consideration of the above, we found that clonazepam can decrease athetoid movement and thus can make conditions of patients better.

A pharmaceutical composition of the present invention may comprise one or more vehicles acceptable for oral administration, for example, diluents (e.g. lactose, dextrose, sucrose, mannitol, sorbitol, cellulose and/or glycine) and lubricants (e.g. silica, talc, stearic acid and magnesium or calcium salts thereof and/or polyethylene glycol), within the limit that effect of the present invention is not affected.

Tablet may also comprise binders such as magnesium aluminum silicate, starch paste, gelatin, tragacanth, methyl cellulose, sodium carboxymethyl cellulose and/or polyvinyl pyrrolidine, and as occasion calls, may comprise disintegrants such as starch, agar, alginic acid or sodium salts thereof or boiling mixtures and/or absorbents, colorants, flavor enhancers and sweeteners. Said tablet can be prepared according to common methods for mixing, granulation or coating.

Therefore, the present invention provides a pharmaceutical composition comprising clonazepam or pharmaceutically acceptable salts thereof and one or more pharmaceutically acceptable vehicles.

The following examples shall explain the invention, but they are only to illustrate the invention and are not intended to limit the same.

### EXAMPLES

A pharmaceutical composition in accordance with the present invention is administered to eight patients with athetoid movement. Concrete scheme of administration and the resulting therapeutic effect depending on symptoms of patients are as follows.

### Example 1

### Male Aged 19 (Body Weight:45 kg), a Mild Athetoid Movement Patient

To a patient who can walk or sit down by himself but has symptom of facial distortions and whose tiptoe is dragged on walking, clonazepam (Roche Korea Co., Rivotril® 0.5 mg) was administered one time a day in the dose of 0.011 mg/kg/day. Facial distortions and symptom of turning the neck were decreased and symptom of dragging the tiptoe was also improved. No tolerance or adverse effect was found.

### Example 2

### Male Aged 36 (Body Weight: 55 kg), a Mild Athetoid Movement Patient

To a patient with mild athetoid movement who has symptom of shaking the hands, clonazepam (Roche Korea Co., Rivotril® 0.5 mg) was administered one time a week in the dose of 0.018 mg/kg/day. Symptom of shaking movement of the hands was improved. No tolerance or adverse effect was found.

### Example 3

### Female Aged 10 (Body Weight: 20 kg), a Severe Athetoid Movement Patient

To a patient with severe athetoid movement who cannot walk or do daily work by herself, clonazepam (Roche Korea Co., Rivotril® 0.5 mg) was administered one time a day in the dose of 0.005 mg/kg/day. Symptom of turning the neck was remarkably decreased, and no deterioration of movement was found.

### Example 4

### Male Aged 24 (Body Weight: 45 kg), a Severe Athetoid Movement Patient

To a patient who cannot walk by himself and feels discomfort due to symptom of turning the body on using computer, clonazepam (Roche Korea Co., Rivotril® 0.5 mg) was administered twice a day in the dose of 0.022 mg/kg/day. Symptom of turning the body was remarkably decreased, and no tolerance or adverse effect was found.

### Example 5

### Female Aged 28 (Body Weight: 50 kg), a Severe Athetoid Movement Patient

To a patient who cannot walk and do a cross-stich by herself, clonazepam (Roche Korea Co., Rivotril® 0.5 mg) was administered one time a day in the dose of 0.012 mg/kg/day. Walking got to be easier when somebody helps the patient to walk, symptom of turning the body was remarkably decreased, and she became able to do a cross-stich by herself. No tolerance or adverse effect was found.

### Example 6

### Male Aged 14 (Body Weight: 17 kg), a Severe Athetoid Movement Patient

To a patient with athetoid movement who cannot walk and sit down by himself and has symptom of turning the neck, clonazepam (Roche Korea Co., Rivotril® 0.5 mg) was administered one time a day in the dose of 0.015 mg/kg/day. He became able to control turning the neck voluntarily.

### Example 7

### Male Aged 14 (Body Weight: 16 kg), a Severe Athetoid Movement Patient

To a patient who cannot walk and sit down by himself and whose neck was extend involuntarily on sitting down on wheelchair, clonazepam (Roche Korea Co., Rivotril® 0.5 mg) was administered one time a day before sleep in the dose of 0.016 mg/kg/day. Symptoms of opening the mouth and extension of the neck were decreased. No tolerance or adverse effect was found.

### Example 8

### Male Aged 17 (Body Weight: 31 kg), a Severe Athetoid Movement Patient

To a patient with athetoid movement who cannot walk and sit down by himself and has rigid hands and feet, clonazepam (Roche Korea Co., Rivotril® 0.5 mg) was administered one time a day in the dose of 0.016 mg/kg/day. The hands and feet became soft and walking was improved.

As discussed above, when a pharmaceutical composition of the present invention is administered, athetoid movement was improved without any tolerance or adverse effect.

### EFFECT OF THE INVENTION

A pharmaceutical composition of the present invention for the treatment of athetoid movement comprising clonazepam can treat effectively athetoisis minimizing tolerance and adverse effect such as sedation.

1. A pharmaceutical composition for the treatment of athetoid movement, comprising clonazepam or pharmaceutically acceptable salt thereof as an active ingredient.

2. The pharmaceutical composition according to claim 1, characterized in that said athetoid movement is manifested by cerebral palsy, lesion of basal ganglia or trauma.

3. The pharmaceutical composition according to claim 1, characterized in that it is administered in the dose of 0.005 to 0.03 mg/kg body weight/day, divided three times a day, and then the dose is controlled depending on the effect of the medicine.

4. The pharmaceutical composition according to claim 1, characterized in that it is administered in the dose of 0.005 to 0.01 mg/kg body weight/day, one time a day, and then the dose is increased by 20 to 40% every three days or every seven days.

5. A method of treating athetoid movement, characterized in that the pharmaceutical composition according to claim 1 is administered in the dose of 0.005 to 0.03 mg/kg body weight/day, divided three times a day, and then the dose is controlled depending on the effect of the medicine.

6. A method of treating athetoid movement, characterized in that the pharmaceutical composition according to claim 1 is administered in the dose of 0.005 to 0.01 mg/kg body weight/day, one time a day, and then the dose is increased by 20 to 40% every three days or every seven days.

7. The method according to claim 5, characterized in that said athetoid movement is manifested by cerebral palsy, lesion of basal ganglia or trauma.

8. The method according to claim 6, characterized in that said athetoid movement is manifested by cerebral palsy, lesion of basal ganglia or trauma.
